(19) United States
(12) Patent Application Publication
(10) Pub. No.: US 2011/0098240 A1
(43) Pub. Date: Apr. 28, 2011

(54) PHARMACEUTICAL COMPOSITION COMPRISING A SGLT2 INHIBITOR IN COMBINATION WITH A DPP-IV INHIBITOR

(75) Inventors: Klaus Dugi, Dresden (DE); Michael Mark, Biberach (DE); Frank Himmelsbach, Mittelbiberach (DE)

(73) Assignee: BOEHRINGER INGELHEIM INTERNATIONAL GMBH, Ingelheim am Rhein (DE)

(21) Appl. No.: 12/673,319

(22) PCT Filed: Aug. 15, 2008
(86) PCT No.: PCT/EP08/60744

§ 371 Date: Apr. 15, 2010

(30) Foreign Application Priority Data
Aug. 16, 2007 (EP) 07114458.8

(51) Int. Cl. A61K 31/7042; A61K 31/7048; A61K 31/7056; A61K 31/5025; A61K 31/52; A61P 3/04; A61K 31/519

(52) U.S. Cl. 514/32; 514/454; 514/248; 514/263.1; 514/265.1

(57) ABSTRACT

The invention relates to a pharmaceutical composition according to claim 1 comprising a SGLT2 inhibitor in combination with a DPP IV inhibitor which is suitable in the treatment or prevention of one or more conditions selected from type 1 diabetes mellitus, type 2 diabetes mellitus, impaired glucose tolerance and hyperglycemia. In addition the present invention relates to methods for preventing or treating of metabolic disorders and related conditions.

### TECHNICAL FIELD OF THE INVENTION

The invention relates to a pharmaceutical composition comprising a SGLT2 inhibitor as described hereinafter in combination with a DPP IV inhibitor as specified hereinafter which is suitable in the treatment or prevention of one or more conditions selected from type 1 diabetes mellitus, type 2 diabetes mellitus, impaired glucose tolerance, impaired fasting blood glucose and hyperglycemia.

Furthermore the invention relates to methods
for preventing, slowing progression of, delaying, or treating a metabolic disorder;
for improving glycemic control and/or for reducing of fasting plasma glucose, of postprandial plasma glucose and/or of glycosylated hemoglobin HbA1c;
for preventing, slowing, delaying or reversing progression from impaired glucose tolerance, impaired fasting blood glucose, insulin resistance and/or from metabolic syndrome to type 2 diabetes mellitus;
for preventing, slowing progression of, delaying or treating of a condition or disorder selected from the group consisting of complications of diabetes mellitus;
for reducing the body weight or preventing an increase of the body weight or facilitating a reduction in body weight;
for preventing or treating the degeneration of pancreatic beta cells and/or for improving and/or restoring the functionality of pancreatic beta cells and/or restoring the functionality of pancreatic insulin secretion;
for preventing, slowing, delaying or treating diseases or conditions attributed to an abnormal accumulation of liver fat;
maintaining and/or improving the insulin sensitivity and/or for treating or preventing hyperinsulinemia and/or insulin resistance,
   in patients in need thereof characterized in that a SGLT2 inhibitor as defined hereinafter is administered in combination or alternation with a DPP IV inhibitor as defined hereinafter.

In addition the present invention relates to the use of a SGLT2 inhibitor as defined hereinafter for the manufacture of a medicament for use in a method as described hereinbefore and hereinafter.

In addition the present invention relates to the use of a DPP IV inhibitor as defined hereinafter for the manufacture of a medicament for use in a method as described hereinbefore and hereinafter.

The invention also relates to a use of a pharmaceutical composition according to this invention for the manufacture of a medicament for use in a method as described hereinbefore and hereinafter.

### BACKGROUND OF THE INVENTION

In the patent applications EP 1 329 456 A1, WO 03/099836, WO 2006/034489, EP 1783122 A1 and EP 1553094 A1 novel compounds are described which possess inhibitory activity on the sodium-dependent glucose cotransporter SGLT2. Therefore the compounds are described as being suitable as inducers of urinary sugar excretion and as medicaments in the treatment of diabetes.

Renal filtration and reuptake of glucose contributes, among other mechanisms, to the steady state plasma glucose concentration and can therefore serve as an antidiabetic target. Reuptake of filtered glucose across epithelial cells of the kidney proceeds via sodium-dependent glucose cotransporters (SGLTs) located in the brush-border membranes in the tubuli along the sodium gradient⁽¹⁾. There are at least 3 SGLT isoforms that differ in their expression pattern as well as in their physico-chemical properties⁽²⁾. SGLT2 is exclusively expressed in the kidney⁽³⁾, whereas SGLT1 is expressed additionally in other tissues like intestine, colon, skeletal and cardiac muscle^{(4,5)}. SGLT3 has been found to be a glucose sensor in interstitial cells of the intestine without any transport function⁽⁶⁾. Potentially, other related, but not yet characterized genes, may contribute further to renal glucose reuptake^{(7,8,9)}. Under normoglycemia, glucose is completely reabsorbed by SGLTs in the kidney, whereas the reuptake capacity of the kidney is saturated at glucose concentrations higher than 10 mM, resulting in glucosuria (“diabetes mellitus”). This threshold concentration can be decreased by SGLT2-inhibition. It has been shown in experiments with the SGLT inhibitor phlorizin that SGLT-inhibition will partially inhibit the reuptake of glucose from the glomerular filtrate into the blood leading to a decrease in blood glucose concentrations and to glucosuria^{(10,11)}.

(1) Wright, E. M. (2001) Am. J. Renal Physiol. 280, F10-F18;

(2) Wright, E. M. et al. (2004) Pflugers Arch. 447(5):510-8;

(3) You, G. et al. (1995) J. Biol. Chem. 270 (49) 29365-29371;

(4) Pajor A M, Wright E M (1992) J Biol. Chem. 267(6):3557-3560;

(5) Zhou, L. et al. (2003) J. Cell. Biochem. 90:339-346;

(6) Diez-Sampedro, A. et al. (2003) Proc. Natl. Acad. Sci. USA 100(20), 11753-11758;

(7) Tabatabai, N. M. (2003) Kidney Int. 64, 1320-1330;

(8) Curtis, R. A. J. (2003) US Patent Appl. 2003/0054453;

(9) Bruss, M. and Bonisch, H. (2001) Cloning and functional characterization of a new human sugar transporter in kidney (Genbank Acc. No. AJ305237);

(10) Rossetti, L. Et al. (987) J. Clin. Invest. 79, 1510-1515;

(11) Gouvea, W. L. (1989) Kidney Int. 35(4):1041-1048.

The compounds Dapagliflozin, Remogliflozin (including Remogliflozin etabonate) and Sergliflozin (including Sergliflozin etabonate) are known as potent SGLT2 inhibitors currently being in development for the treatment of type 2 diabetes mellitus. In the following the chemical structure of said compounds and of further compounds which are described as SGLT2 inhibitors are depicted:

### (1): Dapagliflozin

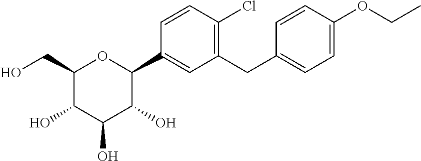

The compound is described for example in WO 03/099836. Crystalline forms are described for example in WO 2008/002824.

### (2): Remogliflozin and Remogliflozin etabonate

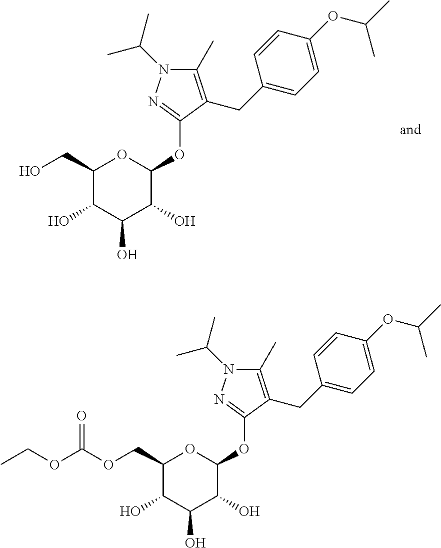

The compound is described for example in EP 1354888 A1.

### (3): Sergliflozin and Sergliflozin etabonate

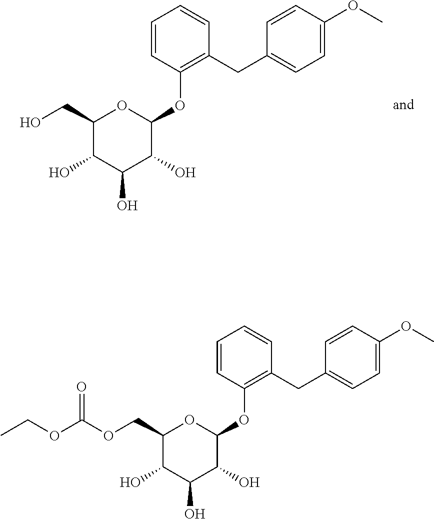

The compounds are described in EP 1 329 456 A1 and a crystalline form of Sergliflozin etabonate is described in EP 1 489 089 A1.

### (4): 1-Chloro-4-(β-D-glucopyranos-1-yl)-2-(4-ethyl-benzyl)-benzene

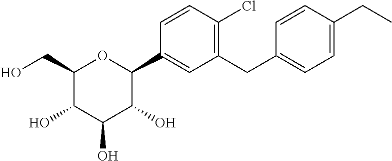

The compound is described in WO 2006/034489.

### (5): (1S)-1,5-anhydro-1-[5-(azulen-2-ylmethyl)-2-hydroxyphenyl]-D-qlucitol

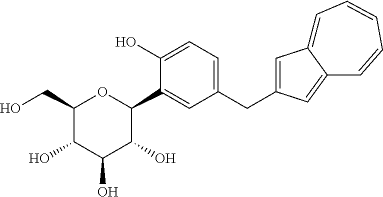

The compound (4-(Azulen-2-ylmethyl)-2-(β-D-glucopyrano-1-yl)-1-hydroxy-benzene) is described in WO 2004/013118 and WO 2006/006496. The crystalline choline salt thereof is described in WO 2007/007628.

### (6): (1S)-1,5-anhydro-1-[3-(1-benzothien-2-ylmethyl)-4-fluorophenyl]-D-glucitol

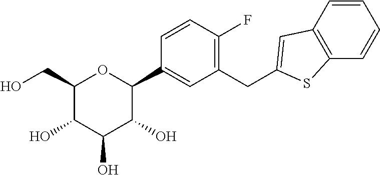

The compound is described in WO 2004/080990 and WO 2005/012326. A cocrystal with L-proline is described in WO 2007/114475.

### (7): Thiophen Derivatives of the Formula (7-1)

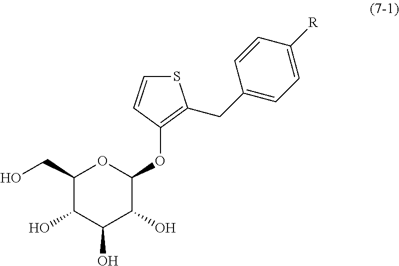

wherein R denotes methoxy or trifluoromethoxy. Such compounds and their method of production are described in WO 2004/007517, DE 102004063099 and WO 2006/072334.

### (8): 1-(β-D-glucopyranosyl)-4-methyl-3-[5-(4-fluorophenyl)-2-thienylmethyl]benzene

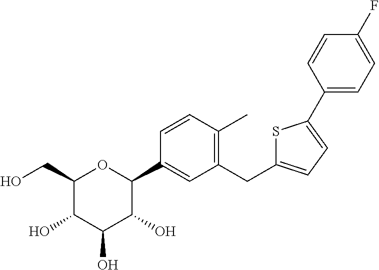

The compound is described in WO 2005/012326. A crystalline hemihydrate is described in WO 2008/069327.

### (9) Spiroketal Derivatives of the Formula (9-1)

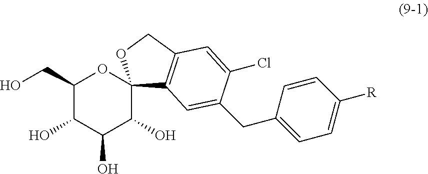

wherein R denotes methoxy, trifluoromethoxy, ethoxy, ethyl, isopropyl or tert. butyl. Such compounds are described in WO 2007/140191 and WO 2008/013280.

DPP IV inhibitors represent a novel class of agents that are being developed for the treatment or improvement in glycemic control in patients with type 2 diabetes.

For example, DPP IV inhibitors and their uses are disclosed in WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769, W02007/014886; WO 2004/050658, WO 2004/111051, WO 2005/058901, WO 2005/097798; WO 2006/068163, WO 2007/071738, WO 2008/017670; WO 2007/054201 or WO 2007/128761.

Type 2 diabetes is an increasingly prevalent disease that due to a high frequency of complications leads to a significant reduction of life expectancy. Because of diabetes-associated microvascular complications, type 2 diabetes is currently the most frequent cause of adult-onset loss of vision, renal failure, and amputations in the industrialized world. In addition, the presence of type 2 diabetes is associated with a two to five fold increase in cardiovascular disease risk.

After long duration of disease, most patients with type 2 diabetes will eventually fail on oral therapy and become insulin dependent with the necessity for daily injections and multiple daily glucose measurements.

The UKPDS (United Kingdom Prospective Diabetes Study) demonstrated that intensive treatment with metformin, sulfonylureas or insulin resulted in only a limited improvement of glycemic control (difference in HbA1c ˜0.9%). In addition, even in patients within the intensive treatment arm glycemic control deteriorated significantly over time and this was attributed to deterioration of β-cell function. Importantly, intensive treatment was not associated with a significant reduction in macrovascular complications, i.e. cardiovascular events.

Therefore there is an unmet medical need for methods, medicaments and pharmaceutical compositions with a good efficacy with regard to glycemic control, with regard to disease-modifying properties and with regard to reduction of cardiovascular morbidity and mortality while at the same time showing an improved safety profile.

Aim of the Present Invention

The aim of the present invention is to provide a pharmaceutical composition and method for preventing, slowing progression of, delaying or treating a metabolic disorder, in particular of type 2 diabetes mellitus.

A further aim of the present invention is to provide a pharmaceutical composition and method for improving glycemic control in a patient in need thereof.

Another aim of the present invention is to provide a pharmaceutical composition and method for preventing, slowing or delaying progression from impaired glucose tolerance (IGT), impaired fasting blood glucose (IFG), insulin resistance and/or metabolic syndrome to type 2 diabetes mellitus.

Yet another aim of the present invention is to provide a pharmaceutical composition and method for preventing, slowing progression of, delaying or treating of a condition or disorder from the group consisting of complications of diabetes mellitus.

A further aim of the present invention is to provide a pharmaceutical composition and method for reducing the weight or preventing an increase of the weight in a patient in need thereof.

Another aim of the present invention is to provide a new pharmaceutical composition with a high efficacy for the treatment of metabolic disorders, in particular of diabetes mellitus, impaired glucose tolerance (IGT), impaired fasting blood glucose (IFG), and/or hyperglycemia, which has good to very good pharmacological and/or pharmacokinetic and/or physicochemical properties.

Further aims of the present invention become apparent to the one skilled in the art by the description hereinbefore and in the following and by the examples.

### SUMMARY OF THE INVENTION

Within the scope of the present invention it has now surprisingly been found that a pharmaceutical composition comprising a SGLT2 inhibitor as defined hereinafter can advantageously be used in combination with a DPP IV inhibitor as specified hereinafter for preventing, slowing progression of, delaying or treating a metabolic disorder, in particular in improving glycemic control in patients. This opens up new therapeutic possibilities in the treatment and prevention of type 2 diabetes mellitus, overweight, obesity, complications of diabetes mellitus and of neighboring disease states.

Therefore in a first aspect the present invention provides a pharmaceutical composition comprising a SGLT2 inhibitor selected from the group consisting of
(1) Dapagliflozin;
(2) Remogliflozin or Remogliflozin etabonate;
(3) Sergliflozin or Sergliflozin etabonate;
(4) 1-Chloro-4-(β-D-glucopyranos-1-yl)-2-(4-ethyl-benzyl)-benzene;
(5) (1S)-1,5-Anhydro-1-[5-(azulen-2-ylmethyl)-2-hydroxyphenyl]-D-glucitol;
(6) (1S)-1,5-Anhydro-1-[3-(1-benzothien-2-ylmethyl)-4-fluorophenyl]-D-glucitol;
(7) Thiophen derivative of the formula (7-1)

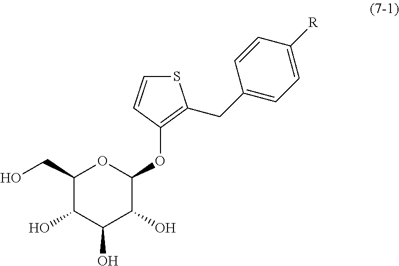

wherein R denotes methoxy or trifluoromethoxy;
(8) 1-(β-D-glucopyranosyl)-4-methyl-3-[5-(4-fluorophenyl)-2-thienylmethyl]benzene;
(9) Spiroketal derivative of the formula (9-1):

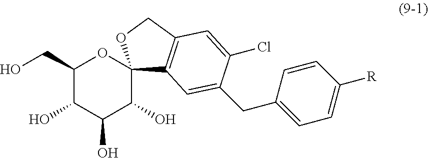

wherein R denotes methoxy, trifluoromethoxy, ethoxy, ethyl, isopropyl or tert. butyl;

or a pharmaceutically acceptable salt, hydrate or solvate thereof;

in combination with a DPP IV inhibitor of formula (I)

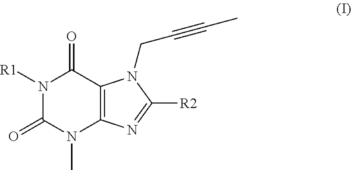

or formula (II)

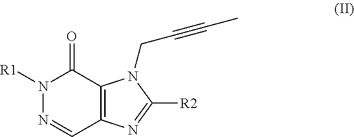

or formula (III)

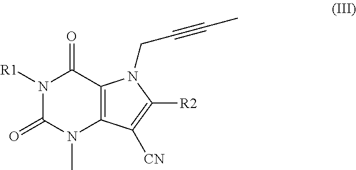

or formula (IV)

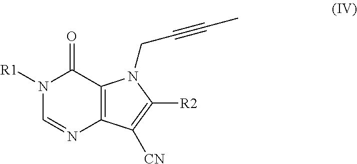

wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3-cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino,

or its pharmaceutically acceptable salt.

According to another aspect of the invention there is provided a method for preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome in a patient in need thereof characterized in that a SGLT2 inhibitor as defined hereinbefore and hereinafter is administered in combination or alternation with a DPP IV inhibitor as defined hereinbefore and hereinafter.

According to another aspect of the invention there is provided a method for improving glycemic control and/or for reducing of fasting plasma glucose, of postprandial plasma glucose and/or of glycosylated hemoglobin HbA1c in a patient in need thereof characterized in that a SGLT2 inhibitor as defined hereinbefore and hereinafter is administered in combination or alternation with a DPP IV inhibitor as defined hereinbefore and hereinafter.

The pharmaceutical composition according to this invention may also have valuable disease-modifying properties with respect to diseases or conditions related to impaired glucose tolerance (IGT), impaired fasting blood glucose (IFG), insulin resistance and/or metabolic syndrome.

According to another aspect of the invention there is provided a method for preventing, slowing, delaying or reversing progression from impaired glucose tolerance (IGT), impaired fasting blood glucose (IFG), insulin resistance and/or from metabolic syndrome to type 2 diabetes mellitus in a patient in need thereof characterized in that a SGLT2 inhibitor as defined hereinbefore and hereinafter is administered in combination or alternation with a DPP IV inhibitor as defined hereinbefore and hereinafter.

As by the use of a pharmaceutical composition according to this invention an improvement of the glycemic control in patients in need thereof is obtainable, also those conditions and/or diseases related to or caused by an increased blood glucose level may be treated.

According to another aspect of the invention there is provided a method for preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease, in a patient in need thereof characterized in that a SGLT2 inhibitor as defined hereinbefore and hereinafter is administered in combination or alternation with a DPP IV inhibitor as defined hereinbefore and hereinafter. The term “tissue ischaemia” particularly comprises diabetic macroangiopathy, diabetic microangiopathy, impaired wound healing and diabetic ulcer.

By the administration of a pharmaceutical composition according to this invention and due to the SGLT2 inhibitory activity excessive blood glucose levels are not converted to insoluble storage forms, like fat, but excreted through the urine of the patient. Therefore no gain in weight or even a reduction of the body weight is the result.

According to another aspect of the invention there is provided a method for reducing the body weight or preventing an increase in body weight or facilitating a reduction in body weight in a patient in need thereof characterized in that a SGLT2 inhibitor as defined hereinbefore and hereinafter is administered in combination or alternation with a DPP IV inhibitor as defined hereinbefore and hereinafter.

The pharmacological effect of the SGLT2 inhibitor in the pharmaceutical composition according to this invention is independent of insulin. Therefore an improvement of the glycemic control is possible without an additional strain on the pancreatic beta cells. By an administration of a pharmaceutical composition according to this invention a beta-cell degeneration and a decline of beta-cell functionality such as for example apoptosis or necrosis of pancreatic beta cells can be delayed or prevented. Furthermore the functionality of pancreatic cells can be improved or restored, and the number and size of pancreatic beta cells increased. It may be shown that the differentiation status and hyperplasia of pancreatic beta-cells disturbed by hyperglycemia can be normalized by treatment with a pharmaceutical composition according to this invention.

According to another aspect of the invention there is provided a method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof characterized in that a SGLT2 inhibitor as defined hereinbefore and hereinafter is administered in combination or alternation with a DPP IV inhibitor as defined hereinbefore and hereinafter.

By the administration of a combination or pharmaceutical composition according to the present invention an abnormal accumulation of fat in the liver may be reduced or inhibited. Therefore according to another aspect of the present invention there is provided a method for preventing, slowing, delaying or treating diseases or conditions attributed to an abnormal accumulation of liver fat in a patient in need thereof characterized in that a SGLT2 inhibitor as defined hereinbefore and hereinafter is administered in combination or alternation with a DPP IV inhibitor as defined hereinbefore and hereinafter. Diseases or conditions which are attributed to an abnormal accumulation of liver fat are particularly selected from the group consisting of general fatty liver, non-alcoholic fatty liver (NAFL), non-alcoholic steatohepatitis (NASH), hyperalimentation-induced fatty liver, diabetic fatty liver, alcoholic-induced fatty liver or toxic fatty liver.

As a result thereof another aspect of the invention provides a method for maintaining and/or improving the insulin sensitivity and/or for treating or preventing hyperinsulinemia and/or insulin resistance in a patient in need thereof characterized in that a SGLT2 inhibitor as defined hereinbefore and hereinafter is administered in combination or alternation with a DPP IV inhibitor as defined hereinbefore and hereinafter.

According to another aspect of the invention there is provided the use of a SGLT2 inhibitor as defined hereinbefore and hereinafter for the manufacture of a medicament for
preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance (IGT), impaired fasting blood glucose (IFG), insulin resistance and/or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease; or
reducing body weight or preventing an increase in body weight or facilitating a reduction in body weight; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of liver fat; or
maintaining and/or improving the insulin sensitivity and/or for treating or preventing hyperinsulinemia and/or insulin resistance;

in a patient in need thereof characterized in that the SGLT2 inhibitor is administered in combination or alternation with a DPP IV inhibitor as defined hereinbefore and hereinafter.

According to another aspect of the invention there is provided the use of a DPP IV inhibitor as defined hereinbefore and hereinafter for the manufacture of a medicament for
preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance (IGT), impaired fasting blood glucose (IFG), insulin resistance and/or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease; or
reducing body weight or preventing an increase in body weight or facilitating a reduction in body weight; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of liver fat; or
maintaining and/or improving the insulin sensitivity and/or for treating or preventing hyperinsulinemia and/or insulin resistance;

in a patient in need thereof characterized in that the DPP IV inhibitor is administered in combination or alternation with a SGLT2 inhibitor as defined hereinbefore and hereinafter.

According to another aspect of the invention there is provided the use of a pharmaceutical composition according to the present invention for the manufacture of a medicament for a therapeutic and preventive method as described hereinbefore and hereinafter.

Definitions

The term “active ingredient” of a pharmaceutical composition according to the present invention means the SGLT2 inhibitor and/or the DPP IV inhibitor according to the present invention.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference>85 cm in men and >90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 110 mg/dL (6.11 mmol/L). The word “fasting” has the usual meaning as a medical term.

The term “hyperglycemia” is defined as the condition in which a subject has a fasting blood glucose concentration above the normal range, greater than 110 mg/dL (6.11 mmol/L). The word “fasting” has the usual meaning as a medical term.

The term “hypoglycemia” is defined as the condition in which a subject has a blood glucose concentration below the normal range of 60 to 115 mg/dL (3.3 to 6.3 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/l), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/l.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio<1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, of al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method it is not possible to distinguish between hepatic and peripheral insulin resistance.

Furthermore insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews of al., Diabetologia 1985, 28: 412-19), of the ratio of intact proinsulin to insulin (Forst et al., Diabetes 2003, 52(Suppl.1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, of al. Diabet Med 1992;9:921-8):

HOMA-IR=[fasting serum insulin (μU/mL)]×[fasting plasma glucose(mmol/L)/22.5]

As a rule, other parameters are used in everyday clinical practice to assess insulin resistance. Preferably, the patient's triglyceride concentration is used, for example, as increased triglyceride levels correlate significantly with the presence of insulin resistance. Patients with a predisposition for the development of IGT or IFG or type 2 diabetes are those having euglycemia with hyperinsulinemia and are by definition, insulin resistant. A typical patient with insulin resistance is usually overweight or obese. If insulin resistance can be detected this is a particularly strong indication of the presence of pre-diabetes. Thus, it may be that in order to maintain glucose homoeostasis a person needs 2-3 times as much insulin as a healthy person, without this resulting in any clinical symptoms.

The methods to investigate the function of pancreatic beta-cells are similar to the above methods with regard to insulin sensitivity, hyperinsulinemia or insulin resistance: An improvement of the beta-cell function can be measured for example by determining a HOMA-index for beta-cell function (Matthews et al., Diabetologia 1985, 28: 412-19), the ratio of intact proinsulin to insulin (Forst et al., Diabetes 2003, 52(Suppl.1): A459), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “pre-diabetes” is the condition wherein an individual is pre-disposed to the development of type 2 diabetes. Pre-diabetes extends the definition of impaired glucose tolerance to include individuals with a fasting blood glucose within the high normal range≧100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes. Insulin resistance can be confirmed in these individuals by calculating the HOMA-IR score. For the purpose of this invention, insulin resistance is defined as the clinical condition in which an individual has a HOMA-IR score>4.0 or a HOMA-IR score above the upper limit of normal as defined for the laboratory performing the glucose and insulin assays.

The term “type 2 diabetes” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002;156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference>40 inches or 102 cm in men, and >35 inches or 94 cm in women; or with regard to a Japanese ethnicity or Japanese patients defined as waist circumference>85 cm in men and ≧90 cm in women;
2. Triglycerides: ≧150 mg/dL
3. HDL-cholesterol<40 mg/dL in men
4. Blood pressure≧130/85 mm Hg (SBP≧130 or DBP≧85)
5. Fasting blood glucose≧110 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor and Diagnose”, TH-Books Verlagsgesellschaft mbH, Frankfurt/Main, 2000.

According to a commonly used definition hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

### DETAILED DESCRIPTION

The aspects according to the present invention, in particular the pharmaceutical compositions, methods and uses, refer to SGLT2 inhibitors as defined hereinbefore and hereinafter.

According to this invention it is to be understood that the definitions of the above listed SGLT2 inhibitors also comprise their pharmaceutically acceptable salts, their hydrates, solvates and polymorphic forms thereof. A preferred SGLT2 inhibitors is Dapagliflozin, including its crystalline forms as described in the WO 2008/002824 which hereby is incorporated by reference in its entirety. Another preferred SGLT2 inhibitor is Remogliflozin including Remogliflozin etabonate.

The aspects according to the present invention, in particular the pharmaceutical compositions, methods and uses, refer to a DPP IV inhibitor as defined hereinbefore and hereinafter, or prodrugs thereof, or pharmaceutically acceptable salts thereof.

Preferred DPP IV inhibitors are any or all of the following compounds and their pharmaceutically acceptable salts:

### (A): 1-[(4-methyl-quinazolin-2-yl)methyl]-3-methyl-7-(2-butyn-1-yl)-8-(3-(R)-amino-piperidin-1-yl)-xanthine (cf. WO 2004/018468, Example 2(142))

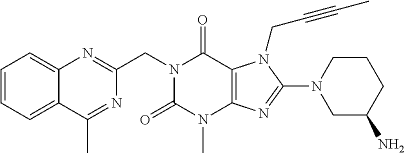

### (B): 1-[([1,5]naphthyridin-2-yl)methyl]-3-methyl-7-(2-butyn-1-yl)-8-((R)-3-amino-piperidin-1-yl)-xanthine (cf. WO 2004/018468, Example 2(252))

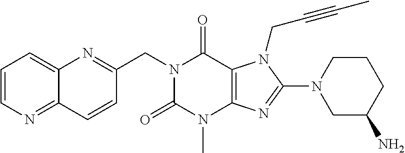

### (C): 1-[(quinazolin-2-yl)methyl]-3-methyl-7-(2-butyn-1-yl)-8-((R)-3-amino-piperidin-1-yl)-xanthine (cf. WO 2004/018468, Example 2(80))

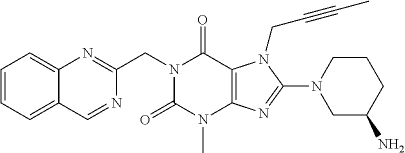

### (D): 2-((R)-3-amino-piperidin-1-yl)-3-(but-2-ynyl)-5-(4-methyl-quinazolin-2-ylmethyl)-3.5-dihydro-imidazo[4,5-d]pyridazin-4-one (cf. WO 2004/050658, Example 136)

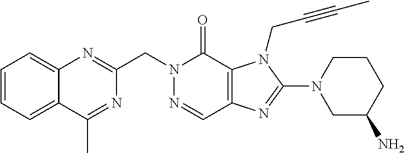

### (E): 1-[(4-methyl-quinazolin-2-yl)methyl]-3-methyl-7-(2-butyn-1-yl)-8-[(2-amino-2-methyl-propyl)-methylamino]-xanthine (cf. WO 2006/029769, Example 2(1))

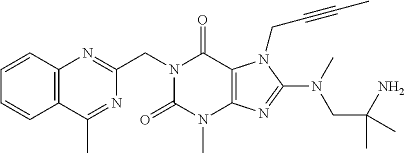

### (F): 1-[(3-cyano-quinolin-2-yl)methyl]-3-methyl-7-(2-butyn-1-yl)-8-((R)-3-amino-piperidin-1-yl)-xanthine (cf. WO 2005/085246, Example 1(30))

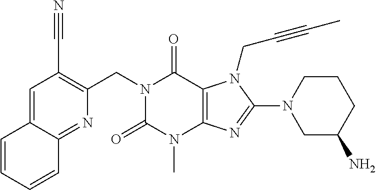

### (G): 1-(2-cyano-benzyl)-3-methyl-7-(2-butyn-1-yl)-8-((R)-3-amino-piperidin-1-yl)-xanthine (cf. WO 2005/085246, Example 1(39))

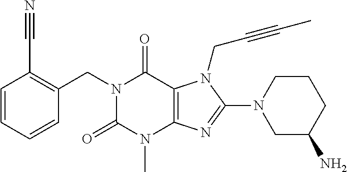

### (H): 1-[(4-methyl-quinazolin-2-yl)methyl]-3-methyl-7-(2-butyn-1-yl)-8-[(S)-(2-amino-propyl)-methylamino]-xanthine (cf. WO 2006/029769, Example 2(4))

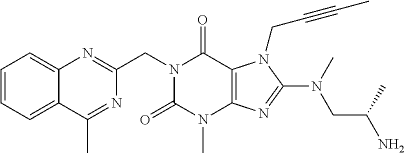

### (I): 1-[(3-cyano-pyridin-2-yl)methyl]-3-methyl-7-(2-butyn-1-yl)-8-((R)-3-amino-piperidin-1-yl)-xanthine (cf. WO 2005/085246, Example 1(52))

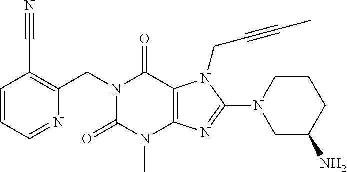

### (J): 1-[(4-methyl-pyrimidin-2-yl)methyl]-3-methyl-7-(2-butyn-1-yl)-8-((R)-3-amino-piperidin-1-yl)-xanthine (cf. WO 2005/085246, Example 1(81))

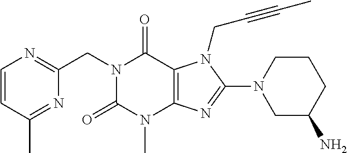

### (K): 1-[(4,6-dimethyl-pyrimidin-2-yl)methyl]-3-methyl-7-(2-butyn-1-yl)-8-((R)-3-amino-piperidin-1-yl)-xanthine (cf. WO 2005/085246, Example 1(82))

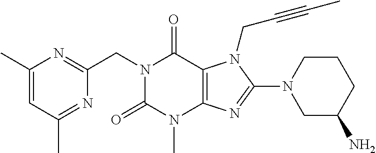

### (L): 1-[(quinoxalin-6-yl)methyl]-3-methyl-7-(2-butyn-1-y)-8-((R)-3-amino-piperidin-1-yl)-xanthine (cf. WO 2005/085246, Example 1(83))

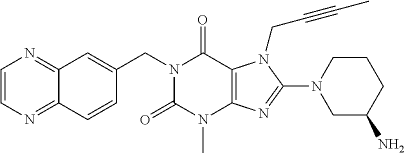

These DPP IV inhibitors are distinguished from structurally comparable DPP IV inhibitors, as they combine exceptional potency and a long-lasting effect with favourable pharmacological properties, receptor selectivity and a favourable side-effect profile or bring about unexpected therapeutic advantages or improvements when combined with other pharmaceutical active substances. Their preparation is disclosed in the publications mentioned.

According to this invention it is to be understood that the definitions of the above listed DPP IV inhibitors also comprise their pharmaceutically acceptable salts as well as hydrates, solvates and polymorphic forms thereof.

The pharmaceutical compositions, methods and uses according to this invention most preferably relate to combinations which are selected from the Table 1.

TABLE 1

| | Compound No. of the SGLT2 | |
|---|---|---|
| No. | inhibitor | DPP IV Inhibitor |
| 1 | (1) | (A) |
| 2 | (1) | (B) |
| 3 | (1) | (C) |
| 4 | (1) | (D) |
| 5 | (1) | (E) |
| 6 | (1) | (F) |
| 7 | (1) | (G) |
| 8 | (1) | (H) |
| 9 | (1) | (I) |
| 10 | (1) | (J) |
| 11 | (1) | (K) |
| 12 | (1) | (L) |
| 13 | (2) | (A) |
| 14 | (2) | (B) |
| 15 | (2) | (C) |
| 16 | (2) | (D) |
| 17 | (2) | (E) |
| 18 | (2) | (F) |
| 19 | (2) | (G) |
| 20 | (2) | (H) |
| 21 | (2) | (I) |
| 22 | (2) | (J) |
| 23 | (2) | (K) |
| 24 | (2) | (L) |
| 25 | (3) | (A) |
| 26 | (3) | (B) |
| 27 | (3) | (C) |
| 28 | (3) | (D) |
| 29 | (3) | (E) |
| 30 | (3) | (F) |
| 31 | (3) | (G) |
| 32 | (3) | (H) |
| 33 | (3) | (I) |
| 34 | (3) | (J) |
| 35 | (3) | (K) |
| 36 | (3) | (L) |
| 37 | (4) | (A) |
| 38 | (4) | (B) |
| 39 | (4) | (C) |
| 40 | (4) | (D) |
| 41 | (4) | (E) |
| 42 | (4) | (F) |
| 43 | (4) | (G) |
| 44 | (4) | (H) |
| 45 | (4) | (I) |
| 46 | (4) | (J) |
| 47 | (4) | (K) |
| 48 | (4) | (L) |
| 49 | (5) | (A) |
| 50 | (5) | (B) |
| 51 | (5) | (C) |
| 52 | (5) | (D) |
| 53 | (5) | (E) |
| 54 | (5) | (F) |
| 55 | (5) | (G) |
| 56 | (5) | (H) |
| 57 | (5) | (I) |
| 58 | (5) | (J) |
| 59 | (5) | (K) |
| 60 | (5) | (L) |
| 61 | (6) | (A) |
| 62 | (6) | (B) |
| 63 | (6) | (C) |
| 64 | (6) | (D) |
| 65 | (6) | (E) |
| 66 | (6) | (F) |
| 67 | (6) | (G) |
| 68 | (6) | (H) |
| 69 | (6) | (I) |
| 70 | (6) | (J) |
| 71 | (6) | (K) |
| 72 | (6) | (L) |
| 73 | (7) | (A) |
| 74 | (7) | (B) |
| 75 | (7) | (C) |
| 76 | (7) | (D) |
| 77 | (7) | (E) |
| 78 | (7) | (F) |
| 79 | (7) | (G) |
| 80 | (7) | (H) |
| 81 | (7) | (I) |
| 82 | (7) | (J) |
| 83 | (7) | (K) |
| 84 | (7) | (L) |
| 85 | (8) | (A) |
| 86 | (8) | (B) |
| 87 | (8) | (C) |
| 88 | (8) | (D) |
| 89 | (8) | (E) |
| 90 | (8) | (F) |
| 91 | (8) | (G) |
| 92 | (8) | (H) |
| 93 | (8) | (I) |
| 94 | (8) | (J) |
| 95 | (8) | (K) |
| 96 | (8) | (L) |
| 97 | (9) | (A) |
| 98 | (9) | (B) |
| 99 | (9) | (C) |
| 100 | (9) | (D) |
| 101 | (9) | (E) |
| 102 | (9) | (F) |
| 103 | (9) | (G) |
| 104 | (9) | (H) |
| 105 | (9) | (I) |
| 106 | (9) | (J) |
| 107 | (9) | (K) |
| 108 | (9) | (L) |

Among the combinations No. 1-108 according to the present invention listed in Table 1, the combinations No. 1, 13, 25, 37, 49, 61, 73, 85 and 97, in particular 1 and 13, are to be emphasized.

The combination of a SGLT2 inhibitor and a DPP IV inhibitor according to this invention significantly improves the glycemic control, in particular in patients as described hereinafter, compared with a monotherapy using either the SGLT2 inhibitor or the DPP IV inhibitor. With monotherapy in a patient, in particular in patients as described hereinafter, the glycemic control can usually not be further improved significantly by an administration of the drug above a certain highest dose. In addition a long term treatment using a highest dose may be unwanted in view of potential side effects. Therefore a full glycemic control cannot be achieved in all patients via a monotherapy using either the SGLT2 inhibitor or the DPP IV inhibitor. In such patients a progression of the diabetes mellitus may continue and complications associated with diabetes mellitus may occur, such as macrovascular complications. The pharmaceutical composition as well as the methods according to the present invention allow a reduction of the HbA1c value to a desired target range, for example <7% and preferably <6.5%, for a higher number of patients compared with a corresponding monotherapy.

In addition the combination of a SGLT2 inhibitor and a DPP IV inhibitor according to this invention allows a reduction in the dose of either the SGLT2 inhibitor or the DPP IV inhibitor or of both active ingredients. A dose reduction is beneficial for patients which otherwise would potentially suffer from side effects in a monotherapy using a higher dose of either the SGLT2 inhibitor or the DPP IV inhibitor. Therefore the pharmaceutical composition as well as the methods according to the present invention show less side effects, thereby making the therapy more tolerable and improving the patients compliance with the treatment.

A monotherapy using a DPP IV inhibitor according to the present invention is not independent from the insulin secretory capacity or the insulin sensitivity of a patient. On the other hand a treatment with the administration of a SGLT2 inhibitor according the present invention does not depend on the insulin secretory capacity or the insulin sensitivity of the patient. Therefore any patient independent of the prevailing insulin levels or insulin resistance and/or hyperinsulinemia may benefit from a therapy using a combination of a SGLT2 inhibitor and a DPP IV inhibitor according to this invention. Independent of their prevailing insulin levels or their insulin resistance or hyperinsulinemia these patients can still be treated with the DPP IV inhibitor because of the combined or alternate administration of the SGLT2 inhibitor.

A DPP IV inhibitor according to the present invention is able—via the increases in active GLP-1 levels—to reduce the glucagon secretion in a patient. This will therefore limit the hepatic glucose production. Furthermore, the elevated active GLP-1 levels produced by the DPP IV inhibitor will have beneficial effects on beta-cell regeneration and neogenesis. All these features of DPP IV inhibitors render a combination with SGLT 2 inhibitors quite useful and therapeutically relevant.

When this invention refers to patients requiring treatment or prevention, it relates primarily to treatment and prevention in humans, but the pharmaceutical composition may also be used accordingly in veterinary medicine on mammals.

As described hereinbefore by the administration of the pharmaceutical composition according to this invention and in particular in view of the high SGLT2 inhibitory activity of the SGLT2 inhibitor therein, excessive blood glucose is excreted through the urine of the patient, so that no gain in weight or even a reduction in body weight may result. Therefore a treatment or prophylaxis according to this invention is advantageously suitable in those patients in need of such treatment or prophylaxis who are diagnosed of one or more of the conditions selected from the group consisting of overweight, class I obesity, class II obesity, class III obesity, visceral obesity and abdominal obesity or for those individuals in which a weight increase is contraindicated.

The pharmaceutical composition according to this invention and in particular the SGLT2 inhibitor therein exhibits a very good efficacy with regard to glycemic control, in particular in view of a reduction of fasting plasma glucose, postprandial plasma glucose and/or glycosylated hemoglobin (HbA1c). By administering a pharmaceutical composition according to this invention, a reduction of HbA1c equal to or greater than preferably 0.5%, even more preferably equal to or greater than 1.0% can be achieved and the reduction is particularly in the range from 1.0% to 1.5%.

Furthermore the method and/or use according to this invention is advantageously applicable in those patients who show one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%.

The present invention also discloses the use of the pharmaceutical composition for improving glycemic control in patients having type 2 diabetes or showing first signs of pre-diabetes. Thus, the invention also includes diabetes prevention. If therefore a pharmaceutical composition according to this invention is used to improve the glycemic control as soon as one of the above-mentioned signs of pre-diabetes is present, the onset of manifest type 2 diabetes mellitus can be delayed or prevented.

Furthermore the pharmaceutical composition according to this invention is particularly suitable in the treatment of patients with insulin dependency, i.e. in patients who are treated or otherwise would be treated or need treatment with an insulin or a derivative of insulin or a substitute of insulin or a formulation comprising an insulin or a derivative or substitute thereof. These patients include patients with diabetes type 2 and patients with diabetes type 1.

It can be found that by using a pharmaceutical composition according to this invention an improvement of the glycemic control can be achieved even in those patients who have insufficient glycemic control in particular despite treatment with an antidiabetic drug, for example despite maximal tolerated dose of oral monotherapy with either metformin or a SGLT2 inhibitor, in particular a SGLT2 inhibitor according to this invention, or a DPP IV inhibitor, in particular a DPP IV inhibitor according to this invention. A maximal tolerated dose with regard to metformin is for example 850 mg three times a day or any equivalent thereof. A maximal tolerated dose with regard to a DPP IV inhibitor according to this invention, in particular with regard to the compound (A) (1-[(4-methyl-quinazolin-2-yl)methyl]-3-methyl-7-(2-butyn-1-yl)-8-(3-(R)-amino-piperidin-1-yl)-xanthine), is for example 10 mg once daily or any equivalent thereof.

In the scope of the present invention the term “insufficient glycemic control” means a condition wherein patients show HbA1c values above 6.5%, in particular above 8%.

Therefore according to a preferred embodiment of the present invention there is provided a method for improving glycemic control and/or for reducing of fasting plasma glucose, of postprandial plasma glucose and/or of glycosylated hemoglobin HbA1c in a patient in need thereof who is diagnosed with impaired glucose tolerance (IGT), impaired fasting blood glucose (IFG) with insulin resistance, with metabolic syndrome and/or with type 2 or type 1 diabetes mellitus characterized in that a SGLT2 inhibitor as defined hereinbefore and hereinafter is administered in combination or alternation with a DPP IV inhibitor as defined hereinbefore and hereinafter.

The lowering of the blood glucose level by the administration of a SGLT2 inhibitor according to this invention is insulin-independent. Therefore a pharmaceutical composition according to this invention is particularly suitable in the treatment of patients who are diagnosed having one or more of the following conditions
insulin resistance,
hyperinsulinemia,
pre-diabetes,
type 2 diabetes mellitus, particular having a late stage type 2 diabetes mellitus,
type 1 diabetes mellitus.

Furthermore a pharmaceutical composition according to this invention is particularly suitable in the treatment of patients who are diagnosed having one or more of the following conditions
(a) obesity (including class I, II and/or III obesity), visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure>130 mm Hg and a diastolic blood pressure≧85 mm Hg,
(e) a fasting blood glucose level>110 mg/dL.

It is assumed that patients diagnosed with impaired glucose tolerance (IGT), impaired fasting blood glucose (IFG), with insulin resistance and/or with metabolic syndrome suffer from an increased risk of developing a cardiovascular disease, such as for example myocardial infarction, coronary heart disease, heart insufficiency, thromboembolic events. A glycemic control according to this invention may result in a reduction of the cardiovascular risks.

A pharmaceutical composition according to this invention, in particular due to the SGLT2-inhibitor therein, exhibits a good safety profile. Therefore a treatment or prophylaxis according to this invention is advantageously possible in those patients for which the mono-therapy with another antidiabetic drug, such as for example metformin, is contraindicated and/or who have an intolerance against such drugs at therapeutic doses. In particular a treatment or prophylaxis according to this invention may be advantageously possible in those patients showing or having an increased risk for one or more of the following disorders: renal insufficiency or diseases, cardiac diseases, cardiac failure, hepatic diseases, pulmonal diseases, catabolytic states and/or danger of lactate acidosis, or female patients being pregnant or during lactation.

Furthermore it can be found that the administration of a pharmaceutical composition according to this invention results in no risk or in a low risk of hypoglycemia. Therefore a treatment or prophylaxis according to this invention is also advantageously possible in those patients showing or having an increased risk for hypoglycemia.

A pharmaceutical composition according to this invention is particularly suitable in the long term treatment or prophylaxis of the diseases and/or conditions as described hereinbefore and hereinafter, in particular in the long term glycemic control in patients with type 2 diabetes mellitus.

The term “long term” as used hereinbefore and hereinafter indicates a treatment of or administration in a patient within a period of time longer than 12 weeks, preferably longer than 25 weeks, even more preferably longer than 1 year.

Therefore a particularly preferred embodiment of the present invention provides a method for therapy, preferably oral therapy, for improvement, especially long term improvement, of glycemic control in patients with type 2 diabetes mellitus, especially in patients with late stage type 2 diabetes mellitus, in particular in patients additionally diagnosed of overweight, obesity (including class I, class II and/or class III obesity), visceral obesity and/or abdominal obesity.

The effects mentioned above are observed both when the SGLT2 inhibitor and the DPP IV inhibitor are administered in combination, for example simultaneously, and when they are administered in alternation, for example successively in separate formulations.

It will be appreciated that the amount of the pharmaceutical composition according to this invention to be administered to the patient and required for use in treatment or prophylaxis according to the present invention will vary with the route of administration, the nature and severity of the condition for which treatment or prophylaxis is required, the age, weight and condition of the patient, concomitant medication and will be ultimately at the discretion of the attendant physician. In general however the SGLT2 inhibitor according to this invention and the DPP IV inhibitor are included in the pharmaceutical composition or dosage form in an amount sufficient that by their administration in combination or alternation the glycemic control in the patient to be treated is improved.

In the following preferred ranges of the amount of SGLT2 inhibitor and of the DPP IV inhibitor to be employed in the pharmaceutical composition and the methods and uses according to this invention are described. These ranges refer to the amounts to be administered per day with respect to an adult patient and can be adapted accordingly with regard to an administration 2, 3, 4 or more times daily and with regard to other routes of administration and with regard to the age of the patient.

Within the scope of the present invention the pharmaceutical composition is preferably administered orally. Other forms of administration are possible and described hereinafter. Preferably the dosage form comprising the SGLT2 inhibitor is administered orally. The route of administration of the DPP IV inhibitor is oral or usually well known.

In general the amount of the SGLT2 inhibitor in the pharmaceutical composition and methods according to this invention is preferably in the range from 1/5 to 1/1 of the amount usually recommended for a monotherapy using said SGLT2 inhibitor. Advantageously, the combination therapy according to the present invention utilizes lower dosages of the individual SGLT2 inhibitor or of the individual DPP IV inhibitor used in monotherapy or used in conventional therapeutics, thus avoiding possible toxicity and adverse side effects incurred when those agents are used as monotherapies.

The amount of the SGLT2 inhibitor is preferably in the range from 0.5 mg to 1000 mg, even more preferably from 5 to 500 mg per day for a human being, for example for approximately 70 kg body weight. With regard to Dapagliflozin a preferred range is from 1 mg to 50 mg, preferably from 2 mg to 30 mg, even more preferably from 1 mg to 10 mg or from 5 mg to 20 mg. Thus particular dosage strengths (e.g. for tablet or capsule) are for example 2.5, 5, 10 mg or 20 mg once a day. With regard to Sergliflozin and Sergliflozin etabonate a preferred range is from 10 mg to 500 mg. Thus particular dosage strengths (e.g. for tablet or capsule) are for example 50, 125, 250 or 500 mg 1, 2 or 3 times daily. With regard to Remogliflozin and Remogliflozin etabonate a preferred range is from 10 mg to 500 mg, preferably from 50 mg to 200 mg or from 100 mg to 400 mg. Thus particular dosage strengths (e.g. for tablet or capsule) are for example 50, 125, 200, 250, 400 or 500 mg 1, 2 or 3 times daily. The oral administration is preferred. Therefore a pharmaceutical composition may comprise the hereinbefore mentioned amounts.

In general the amount of the DPP IV inhibitor in the pharmaceutical composition and methods according to this invention is preferably in the range from 1/5 to 1/1 of the amount usually recommended for a monotherapy using said DPP IV inhibitor.

Typically, the dosage required of the DPP IV inhibitors mentioned herein when administered intravenously is 0.1 mg to 10 mg, preferably 0.25 mg to 5 mg, and when administered orally 0.5 mg to 100 mg, preferably 2.5 mg to 50 mg, or 0.5 mg to 10 mg, more preferably 2.5 mg to 10 mg or 1 mg to 5 mg, in each case 1 to 4 times a day. Thus, the dosage required of the compound (A) (1-[(4-methyl-quinazolin-2-yl)methyl]-3-methyl-7-(2-butyn-1-yl)-8-(3-(R)-amino-piperidin-1-yl)-xanthine) when administered orally is 0.5 mg to 10 mg per patient per day, preferably 2.5 mg to 10 mg per patient per day (more preferably 5 mg to 10 mg per patient per day) or 1 mg to 5 mg per patient per day.

A dosage form prepared with a pharmaceutical composition comprising a DPP IV inhibitor mentioned herein contain the active ingredient in a dosage range of 0.1-100 mg. Particular dosages are 0.5 mg, 1 mg, 2.5 mg, 5 mg and 10 mg. Thus, particular dosage strengths of the compound (A) (1-[(4-methyl-quinazolin-2-yl)methyl]-3-methyl-7-(2-butyn-1-yl)-8-(3-(R)-amino-piperidin-1-yl)-xanthine) are 0.5 mg, 1 mg, 2.5 mg, 5 mg and 10 mg, more particular dosage strengths thereof are 1 mg, 2.5 mg and 5 mg.

The amount of the SGLT2 inhibitor and of the DPP IV inhibitor in the pharmaceutical composition according to this invention correspond to the respective dosage ranges as provided hereinbefore. For example a pharmaceutical composition comprises an amount of 1 to 50 mg, preferably from 2 to 10 mg, of the compound (1) and of the compound (A) (1-[(4-methyl-quinazolin-2-yl)methyl]-3-methyl-7-(2-butyn-1-yl)-8-(3-(R)-amino-piperidin-1-yl)-xanthine) in an amount of 0.5 mg to 10 mg.

In the methods and uses according to the present invention the SGLT2 inhibitor and the DPP IV inhibitor are administered in combination or alternation. The term “administration in combination” means that both active ingredients are administered at the same time, i.e. simultaneously, or essentially at the same time. The term “administration in alternation” means that at first a first active ingredient is administered and after a period of time the second active ingredient is administered, i.e. both active ingredients are administered sequentially. The period of time may be in the range from 30 min to 12 hours. The administration which is in combination or in alternation may be once, twice, three times or four times daily.

With regard to the administration of the SGLT2 inhibitor in combination with the DPP IV inhibitor both active ingredients may be present in a single dosage form, for example in a tablet or capsule, or each active ingredient may be present in a separate dosage form, for example in two different or identical dosage forms.

With regard to their administration in alternation each of the active ingredients is present in a separate dosage form, for example in two different or identical dosage forms.

Therefore the pharmaceutical composition according to this invention may be present as single dosage forms which comprise both the SGLT2 inhibitor and the DPP IV inhibitor as well as separate dosage forms wherein one dosage form comprises the SGLT2 inhibitor and the other dosage form comprises the DPP IV inhibitor.

The case may arise in which one active ingredient has to be administered more often, for example twice per day, than the other active ingredient, which for example needs administration once daily. Therefore the term “administration in combination or alternation” also includes an administration scheme in which first both active ingredients are administered in combination or alternation and after a period of time only one active ingredient is administered again or vice versa.

Therefore the present invention also includes pharmaceutical compositions which are present a separate dosage forms wherein one dosage form comprises the SGLT2 inhibitor and the DPP IV inhibitor and the other dosage form comprises either the SGLT2 inhibitor or the DPP IV inhibitor.

A pharmaceutical composition which is present as a separate or multiple dosage form, preferably as a kit of parts, is useful in combination therapy to flexibly suit the individual therapeutic needs of the patient.

A preferred kit of parts comprises
(a) a first containment containing a dosage form comprising the SGLT2 inhibitor and at least one pharmaceutically acceptable carrier, and
(b) a second containment containing a dosage form comprising the DPP IV inhibitor and at least one pharmaceutically acceptable carrier.

A further aspect of the present invention is a manufacture comprising the pharmaceutical composition being present as separate dosage forms according to the present invention and a label or package insert comprising instructions that the separate dosage forms are to be administered in combination or alternation.

A yet further aspect of the present invention is a manufacture comprising a medicament which comprises a SGLT2 inhibitor according to the present invention and a label or package insert which comprises instructions that the medicament may or is to be administered in combination or alternation with a medicament comprising a DPP IV inhibitor according to the present invention.

Another further aspect of the present invention is a manufacture comprising a medicament which comprises a DPP IV inhibitor according to the present invention and a label or package insert which comprises instructions that the medicament may or is to be administered in combination or alternation with a medicament comprising a SGLT2 inhibitor according to the present invention.

The desired dose of the pharmaceutical composition according to this invention may conveniently be presented in a once daily or as divided dose administered at appropriate intervals, for example as two, three or more doses per day.

The pharmaceutical composition may be formulated for oral, rectal, nasal, topical (including buccal and sublingual), transdermal, vaginal or parenteral (including intramuscular, sub-cutaneous and intravenous) administration in liquid or solid form or in a form suitable for administration by inhalation or insufflation. Oral administration is preferred. The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition and the dosage forms preferably comprises one or more pharmaceutical acceptable carriers which must be “acceptable” in the sense of being compatible with the other ingredients of the formulation and not deleterious to the recipient thereof.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

The pharmaceutical compositions and methods according to this invention show advantageous effects in the treatment and prevention of those diseases and conditions as described hereinbefore compared with pharmaceutical compositions and methods which comprise only one of both active ingredients. Advantageous effects may be seen for example with respect to efficacy, dosage strength, dosage frequency, pharmacodynamic properties, pharmacokinetic properties, fewer adverse effects, etc.

Examples of pharmaceutically acceptable carriers are known to the one skilled in the art.

Methods for the manufacture of SGLT2 inhibitors according to this invention are known to the one skilled in the art. Preferred methods are described for example in the literature and patent applications as cited in the chapter “Background of the invention”.

The methods of synthesis for the DPP IV inhibitors according to this invention are known to the skilled person. Advantageously the DPP IV inhibitors according to this invention can be prepared using synthetic methods as described in the literature. Thus, for example, purine derivatives of formula (I) can be obtained as described in WO 2002/068420, WO 2004/018468, WO 2005/085246, WO 2006/029769 or WO 2006/048427, the disclosures of which are incorporated herein. Purine derivatives of formula (II) can be obtained as described, for example, in WO 2004/050658 or WO 2005/110999, the disclosures of which are incorporated herein. Purine derivatives of formula (III) and (IV) can be obtained as described, for example, in WO 2006/068163, WO 2007/071738 or WO 2008/017670, the disclosures of which are incorporated herein. The preparation of those DPP IV inhibitors, which are specifically mentioned hereinabove, is disclosed in the publications mentioned in connection therewith. Polymorphous crystal modifications and formulations of particular DPP IV inhibitors are disclosed in WO 2007/054201 and WO 2007/128724, respectively, the disclosures of which are incorporated herein in their entireties.

The DPP IV inhibitor may be present in the form of a pharmaceutically acceptable salt. Pharmaceutically acceptable salts include such as salts of inorganic acid like hydrochloric acid, sulfuric acid and phosphoric acid; salts of organic carboxylic acid like oxalic acid, acetic acid, citric acid, malic acid, benzoic acid, maleic acid, fumaric acid, tartaric acid, succinic acid and glutamic acid and salts of organic sulfonic acid like methanesulfonic acid and p-toluenesulfonic acid. The salts can be formed by combining the compound and an acid in the appropriate amount and ratio in a solvent and decomposer. They can be also obtained by the cation or anion exchange from the form of other salts.

The SGLT2 inhibitor and/or the DPP IV inhibitor or a pharmaceutically acceptable salt thereof may be present in the form of a solvate such as a hydrate or alcohol adduct.

Any of the above mentioned combinations and methods within the scope of the invention may be tested by animal models known in the art. In the following in vivo experiments are described which are suitable to evaluate pharmacologically relevant properties of pharmaceutical compositions and methods according to this invention:

Pharmaceutical compositions and methods according to this invention can be tested in genetically hyperinsulinemic or diabetic animals like db/db mice, ob/ob mice, Zucker Fatty (fa/fa) rats or Zucker Diabetic Fatty (ZDF) rats. In addition, they can be tested in animals with experimentally induced diabetes like HanWistar or Sprague Dawley rats pretreated with streptozotocin.

The effect on glycemic control of the combinations according to this invention can be tested after single dosing of a SGLT2 inhibitor and a DPP IV inhibitor alone and in combination in an oral glucose tolerance test in the animal models described hereinbefore. The time course of blood glucose is followed after on oral glucose challenge in overnight fasted animals. The combinations according to the present invention significantly improve glucose excursion compared to each monotherapy as measured by reduction of peak glucose concentrations or reduction of glucose AUC. In addition, after multiple dosing of a SGLT2 inhibitor and a DPP IV inhibitor alone and in combination in the animal models described hereinbefore, the effect on glycemic control can be determined by measuring the HbA1c value in blood. The combinations according to this invention significantly reduce HbA1c compared to each monotherapy.

The possible dose reduction of either the SGLT2 inhibitor or the DPP-IV inhibitor or of both active ingredients can be tested by the effect on glycemic control of lower doses of the combinations and monotherapies in the animal models described hereinbefore. The combinations according to this invention at the lower doses significantly improve glycemic control compared to placebo treatment whereas the monotherapies at lower doses do not.

The improved independence from insulin of the treatment according to this invention can be shown after single dosing in oral glucose tolerance tests in the animal models described hereinbefore. The time course of plasma insulin is followed after a glucose challenge in overnight fasted animals. The SGLT2 inhibitor in combination with the DPP IV inhibitor will exhibit lower insulin peak concentrations or insulin AUC at lower blood glucose excursion than the DPP IV inhibitor alone.

The increase in active GLP-1 levels by treatment according to this invention after single or multiple dosing can be determined by measuring those levels in the plasma of animal models described hereinbefore in either the fasting or postprandial state. Likewise, a reduction in glucagon levels in plasma can be measured under the same conditions. The SGLT2 inhibitor in combination with the DPP IV inhibitor will exhibit higher active GLP-1 concentrations and lower glucagon concentrations than the SGLT2 inhibitor alone.

A superior effect of the combination of a SGLT2 inhibitor and a DPP IV inhibitor according to the present invention than of the SGLT2 inhibitor alone on beta-cell regeneration and neogenesis can be determined after multiple dosing in the animal models described hereinbefore by measuring the increase in pancreatic insulin content, or by measuring increased beta-cell mass by morphometric analysis after immunhistochemical staining of pancreatic sections, or by measuring increased glucose-stimulated insulin secretion in isolated pancreatic islets.

The Examples that follow are intended to illustrate the present invention without restricting it.

### PHARMACOLOGICAL EXAMPLE

The following example show the beneficial effect on glycemic control of the combination of a SGLT2 inhibitor and a DPP IV inhibitor according to the present invention as compared to the respective monotherapies. All experimental protocols concerning the use of laboratory animals are reviewed by a federal Ethics Committee and approved by governmental authorities. According to this example an oral glucose tolerance test is performed in overnight fasted male Sprague Dawley rats (Crl:CD(SD)) with a body weight of about 200 g. A pre-dose blood sample is obtained by tail bleed. Blood glucose is measured with a glucometer, and the animals are randomized for blood glucose (n=5/group). Subsequently, the groups receive a single oral administration of either vehicle alone (0.5% aqueous hydroxyethylcellulose containing 0.015% Polysorbat 80) or vehicle containing either the SGLT2 inhibitor or the DPPIV inhibitor or the combination of the SGLT2 inhibitor with the DPPIV inhibitor. The animals receive an oral glucose load (2 g/kg) 30 min after compound administration. Blood glucose is measured in tail blood 30 min, 60 min, 90 min, and 120 min after the glucose challenge. Glucose excursion is quantified by calculating the reactive glucose AUC. The data are presented as mean±S.E.M. Statistical comparisons are conducted by Student's t test.

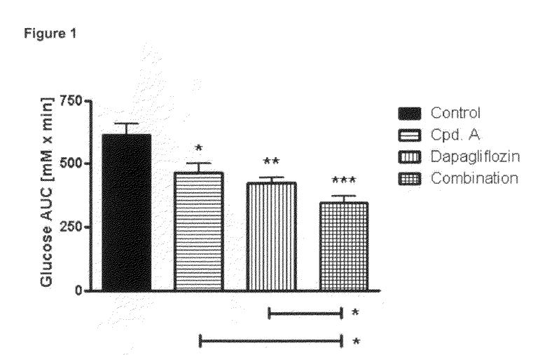

The result is shown in FIG. 1. “Cpd. A” is the DPP IV inhibitor 1-[(4-methyl-quinazolin-2-yl)methyl]-3-methyl-7-(2-butyn-1-yl)-8-(3-(R)-amino-piperidin-1-yl)-xanthine and is administered at a dose of 1 mg/kg. Dapagliflozin is the SGLT2 inhibitor and is administered at a dose of 0.3 mg/kg. In the combination, the DPP IV inhibitor and dapagliflozin are administered together at the same doses as in the respective monotherapies. P values versus control are indicated by symbols above the bars. P values of the combination versus the monotherapies are indicated below the figure (*, p<0.05; **, p<0.01; ***, p<0.001). The DPP IV inhibitor reduces glucose excursion by 25%, and dapagliflozin reduces glucose excursion by 31% in these non-diabetic animals. The combination decreases glucose excursion in the oral glucose tolerance test by 44%, and this reduction in glucose AUC is statistically significant versus each monotherapy.

### EXAMPLES OF FORMULATIONS

The following examples of formulations, which may be obtained analogously to methods known in the art, serve to illustrate the present invention more fully without restricting it to the contents of these examples. The term “active substance” denotes one or more compounds according to the invention, i.e. denotes a SGLT2 inhibitor according to this invention or a DPP IV inhibitor according to this invention or a combination of said SGLT2 inhibitor with said DPP IV inhibitor, for example selected from the combinations 1 to 108 as listed in Table 1. Additional suitable formulations for the DPP IV inhibitors may be those formulations disclosed in the application WO 2007/128724, the disclosure of which is incorporated herein in its entirety.

### Example 1

### Dry Ampoule Containing 75 mg of Active Substance per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use, the product is dissolved in water for injections.

### Example 2

### Dry Ampoule Containing 35 mg of Active Substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried. To produce the solution ready for use, the product is dissolved in water for injections.

### Example 3

### Tablet Containing 50 mg of Active Substance

Composition:

| | | |
|---|---|---|
| (1) Active substance | 50.0 | mg |
| (2) Lactose | 98.0 | mg |
| (3) Maize starch | 50.0 | mg |
| (4) Polyvinylpyrrolidone | 15.0 | mg |
| (5) Magnesium stearate | 2.0 | mg |
| | 215.0 | mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### Example 4

### Tablet Containing 350 mg of Active Substance

Preparation:

| | | |
|---|---|---|
| (1) Active substance | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### Example 5

### Capsules Containing 50 mg of Active Substance

Composition:

| | | |
|---|---|---|
| (1) Active substance | 50.0 | mg |
| (2) Dried maize starch | 58.0 | mg |
| (3) Powdered lactose | 50.0 | mg |
| (4) Magnesium stearate | 2.0 | mg |
| | 160.0 | mg |

Preparation: (1) is triturated with (3). This trituration is added to the mixture of (2) and (4) with vigorous mixing. This powder mixture is packed into size 3 hard gelatin capsules in a capsule filling machine.

Example 6: Capsules containing 350 mg of active substance

Composition:

| | | |
|---|---|---|
| (1) Active substance | 350.0 | mg |
| (2) Dried maize starch | 46.0 | mg |
| (3) Powdered lactose | 30.0 | mg |
| (4) Magnesium stearate | 4.0 | mg |
| | 430.0 | mg |

Preparation:

(1) is triturated with (3). This trituration is added to the mixture of (2) and (4) with vigorous mixing. This powder mixture is packed into size 0 hard gelatin capsules in a capsule filling machine.

1. A pharmaceutical composition comprising a SGLT2 inhibitor selected from the group consisting of
(1) Dapagliflozin;
(2) Remogliflozin or Remogliflozin etabonate;
(3) Sergliflozin or Sergliflozin etabonate;
(4) 1-Chloro-4-(β-D-glucopyranos-1-yl)-2-(4-ethyl-benzyl)-benzene;
(5) (1S)-1,5-Anhydro-1-[5-(azulen-2-ylmethyl)-2-hydroxyphenyl]-D-glucitol;
(6) (1S)-1,5-Anhydro-1-[3-(1-benzothien-2-ylmethyl)-4-fluorophenyl]-D-glucitol;
(7) Thiophen derivative of the formula (7-1) 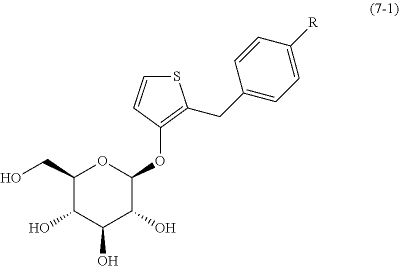
wherein R denotes methoxy or trifluoromethoxy;
(8) 1-(β-D-glucopyranosyl)-4-methyl-3-[5-(4-fluorophenyl)-2-thienylmethyl]benzene;
(9) Spiroketal derivative of the formula (9-1): 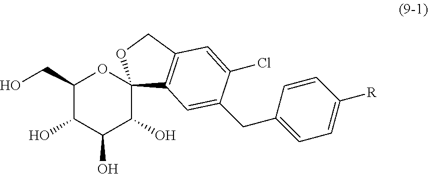
wherein R denotes methoxy, trifluoromethoxy, ethoxy, ethyl, isopropyl or tert. butyl;
or a pharmaceutically acceptable salt, hydrate or solvate thereof;
in combination with a DPP IV inhibitor of formula (I) 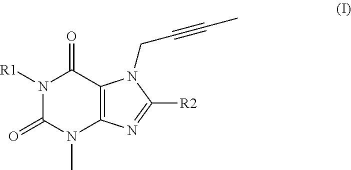
or formula (II) 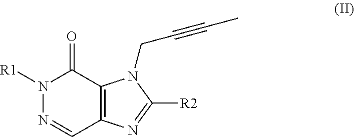
or formula (III) 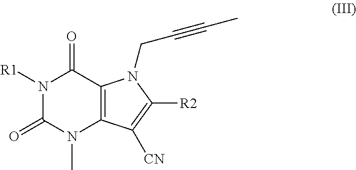
or formula (IV) 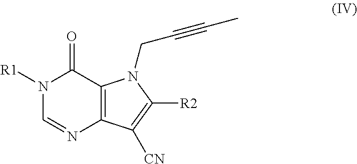
wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3 -cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino,
or its pharmaceutically acceptable salt.

2. The pharmaceutical composition according to claim 1 wherein the DPP IV inhibitor is selected from the group consisting of
1-[(4-methyl-quinazolin-2-yl)methyl]-3-methyl-7-(2-butyn-1-yl)-8-(3-(R)-amino-piperidin-1-yl)-xanthine,
1-[([1,5]naphthyridin-2-yl)methyl]-3-methyl-7-(2-butyn-1-yl)-8-((R)-3-amino-piperidin-1-yl)-xanthine,
1-[(quinazolin-2-yl)methyl]-3-methyl-7-(2-butyn-1-yl)-8-((R)-3-amino-piperidin-1-yl)-xanthine,
2-((R)-3-amino-piperidin-1-yl)-3-(but-2-ynyl)-5-(4-methyl-quinazolin-2-ylmethyl)-3.5-dihydro-imidazo[4,5-d]pyridazin-4-one,
1-[(4-methyl-quinazolin-2-yl)methyl]-3-methyl-7-(2-butyn-1-yl)-8-[(2-amino-2-methyl-propyl)-methylamino]-xanthine,
1-[(3-cyano-quinolin-2-yl)methyl]-3-methyl-7-(2-butyn-1-yl)-8-((R)-3-amino-piperidin-1-yl)-xanthine,
1-(2-cyano-benzyl)-3-methyl-7-(2-butyn-1-yl)-8-((R)-3-amino-piperidin-1-yl)-xanthine,
1-[(4-methyl-quinazolin-2-yl)methyl]-3-methyl-7-(2-butyn-1-yl)-8-[(S)-(2-amino-propyl)-methylamino]-xanthine,
1-[(3-cyano-pyridin-2-yl)methyl]-3-methyl-7-(2-butyn-1-yl)-8-((R)-3-amino-piperidin-1-yl)-xanthine,
1-[(4-methyl-pyrimidin-2-yl)methyl]-3-methyl-7-(2-butyn-1-yl)-8-((R)-3-amino-piperidin-1-yl)-xanthine,
1-[(4,6-dimethyl-pyrimidin-2-yl)methyl]-3-methyl-7-(2-butyn-1-yl)-8-((R)-3-amino-piperidin-1-yl)-xanthine and
1-[(quinoxalin-6-yl)methyl]-3-methyl-7-(2-butyn-1-yl)-8-((R)-3-amino-piperidin-1-yl)-xanthine,
or a pharmaceutically acceptable salt thereof.

3. The pharmaceutical composition according to claim 1 characterized in that the composition is suitable for combined or simultaneous or sequential use of the SGLT2 inhibitor and the DPP IV inhibitor.

4. The pharmaceutical composition according to claim 1 characterized in that the SGLT2 inhibitor and the DPP IV inhibitor are present in a single dosage form.

5. The pharmaceutical composition according to claim 1 characterized in that the SGLT2 inhibitor and the DPP IV inhibitor are present each in a separate dosage form.

6. Method for preventing, slowing the progression of, delaying or treating a metabolic disorder selected from the group consisting of type 1 diabetes mellitus, type 2 diabetes mellitus, impaired glucose tolerance, impaired fasting blood glucose, hyperglycemia, postprandial hyperglycemia, overweight, obesity and metabolic syndrome in a patient in need thereof characterized in that a SGLT2 inhibitor according to claim 1 is administered in combination or alternation with a DPP IV inhibitor according to claim 1.

7. Method for improving glycemic control and/or for reducing of fasting plasma glucose, of postprandial plasma glucose and/or of glycosylated hemoglobin HbA1c in a patient in need thereof characterized in that a SGLT2 inhibitor according to claim 1 is administered in combination or alternation with a DPP IV inhibitor according to claim 1.

8. Method for preventing, slowing, delaying or reversing progression from impaired glucose tolerance, impaired fasting blood glucose, insulin resistance and/or from metabolic syndrome to type 2 diabetes mellitus in a patient in need thereof characterized in that a SGLT2 inhibitor according to claim 1 is administered in combination or alternation with a DPP IV inhibitor according to claim 1.

9. Method for preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease, in a patient in need thereof characterized in that a SGLT2 inhibitor according to claim 1 is administered in combination or alternation with a DPP IV inhibitor according to claim 1.

10. Method for reducing body weight or preventing an increase in body weight or facilitating a reduction in body weight in a patient in need thereof characterized in that a SGLT2 inhibitor according to claim 1 is administered in combination or alternation with a DPP IV inhibitor according to claim 1.

11. Method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof characterized in that a SGLT2 inhibitor according to claim 1 is administered in combination or alternation with a DPP IV inhibitor according to claim 1.

12. Method for preventing, slowing, delaying or treating diseases or conditions attributed to an abnormal accumulation of liver fat in a patient in need thereof characterized in that a SGLT2 inhibitor according to claim 1 is administered in combination or alternation with a DPP IV inhibitor according to claim 1.

13. Method for maintaining and/or improving the insulin sensitivity and/or for treating or preventing hyperinsulinemia and/or insulin resistance in a patient in need thereof characterized in that a SGLT2 inhibitor according to claim 1 is administered in combination or alternation with a DPP IV inhibitor according to claim 1.

14. (canceled)

15. (canceled)

16. (canceled)

17. Method according to claim 6 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

18. (canceled)

19. (canceled)

20. (canceled)

21. (canceled)

22. (canceled)

23. Method according to claim 7 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

24. Method according to claim 8 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

25. Method according to claim 9 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

26. Method according to claim 10 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

27. Method according to claim 11 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

28. Method according to claim 12 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.

29. Method according to claim 13 wherein the patient is:
(1) an individual diagnosed of one or more of the conditions selected from the group consisting of overweight, obesity, visceral obesity and abdominal obesity; or
(2) an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%; or
(3) an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL; or
(4) an individual for whom the monotherapy with metformin is contraindicated and/or who has an intolerance against metformin at therapeutic doses; or
(5) an individual with insufficient glycemic control despite monotherapy with a SGLT2 inhibitor; or
(6) an individual with insufficient glycemic control despite monotherapy with a DPP IV inhibitor.
